(19) United States
(12) Patent Application Publication
(10) Pub. No.: US 2015/0025809 A1
(43) Pub. Date: Jan. 22, 2015

(54) Method of Regulating Caloric Intake

(71) Applicants: Tina M. Herron, Dunedin, FL (US); Patrick D. Herron, Dunedin, FL (US)

(72) Inventors: Tina M. Herron, Dunedin, FL (US); Patrick D. Herron, Dunedin, FL (US)

(21) Appl. No.: 13/947,081

(22) Filed: Jul. 21, 2013

(51) Int. Cl. A61B 5/00

(52) U.S. Cl. CPC A61B 5/4866
USPC 702/19

(57) ABSTRACT

A method, including: determining steady state values for daily caloric parameters that conform a person to weight management goal, the daily caloric parameters including a daily base metabolic rate (BMR) and a target offset from a daily total daily energy expenditure (TDEE), wherein the TDEE includes the BMR; determining an intake shortfall for a plurality of days between actual daily offsets and the steady state target offset; making a recommendation including food to be consumed to reduce the shortfall, the recommendation effective to account for a transient change in a hunger response resulting from the consumption of the recommended food; causing consumption on an up adjustment day; and determining transient values for at least one of the daily caloric parameters to be used subsequent to the consumption on the up-adjustment day effective to bring an average of the actual daily offsets closer to the steady state target offset.

### CROSS REFERENCE TO RELATED APPLICATIONS

This application is a continuation-in-part of pending U.S. patent application Ser. No. 13/840,175 filed on 15 Mar. 2013, which claims the benefit of U.S. Provisional Application 61/698,297, filed 7 Sep. 2012.

### FIELD OF THE INVENTION

The invention is related to regulating caloric intake. In particular, the invention is related to periodically correcting any caloric shortfalls from a target offset of the total daily energy expenditure.

### BACKGROUND OF THE INVENTION

Weight loss diet plans traditionally specify a maximum caloric intake, and may further regulate the composition of the foods to maximize health benefits obtainable given the maximum caloric intake restriction. In certain instances the weight loss diet plan will specify foods that are compatible with the diet, and foods that are incompatible with the diet. Whether or not a food is compatible with the diet may be spelled out by, for example, a list of compatible or incompatible foods. Alternately, compatibility may not be so clearly defined, but instead may be derived from one or more sets of criteria. For example, foods that have a high ratio of calories to health benefits, such as nutritional value, may be considered “empty calorie” foods. The ratio may or may not be set definitively, but certain foods are essentially considered empty calorie foods. Examples of empty calorie foods include, but are not limited to, cookies, candy, ice cream, soft drinks, refined grains, butter, steroids, and alcoholic beverages. Unfortunately, there often exists a strong affinity for such empty calorie foods within the person on the weight loss diet plan. Other dietary restrictions may also be present and may impact the foods that the person on the diet may be permitted. For example, health conditions may force a low sodium intake requirement, a high iron intake requirement, or a low gluten requirement etc.

In light of the phenomenon of weight loss diet burn out, where a person on a diet begins to lose motivation to remain on the diet, some diet plans allow for a caloric “spike day,” or a caloric “cheat day.” During a spike day the person on the diet may be allowed to exceed the usual maximum caloric intake, but may still be restricted to food items compatible with the goals of the diet. An example of a diet having one or more spike days may be a “Zigzag Diet.” In this diet plan the person on the diet is limited to a maximum caloric intake below their Total Daily Energy Expenditure (TDEE), which is the amount of calories a body needs to function at a certain activity level in a day, for several days. Following this, for a few days the person is allowed a greater caloric intake for a few days, where the greater caloric intake may be at or above the TDEE. This is meant to prevent the metabolism from slowing down in response to a consistent reduction in caloric intake. Such a slowing down of the metabolism would reduce the rate of weight loss. Under the zigzag diet, while the maximum daily caloric intake varies over a period of several days that make up one cycle of relatively low and relatively high maximum caloric intake days, on average, over the life of the cycle, and for each cycle, the average maximum daily caloric intake remains the same.

During a cheat day the weight loss diet plan may essentially be suspended for a day, and the person on the diet is allowed to exceed the maximum caloric intake for the cheat day with whatever type of food they wish, within the health guidelines of the diet. In this situation, that which occurs on the cheat day is essentially ignored and the diet resumes after the carnage.

### BRIEF DESCRIPTION OF THE DRAWINGS

The invention is explained in the following description in view of the drawings that show:

FIG. 1 is a flow chart showing steps of an exemplary embodiment of the method disclosed herein.

FIG. 2 is a chart of several example favored food items and associated nutrition information.

FIG. 3 is a flow chart showing steps of an alternate exemplary embodiment of the method disclosed herein.

FIG. 4 is a chart showing an example of consumption of an item of interest over time.

FIG. 5 is a graphical representation of the amount of the item of interest consumed and a severity of a symptom over time of FIG. 4.

FIG. 6 shows a graphical representation of FIG. 5 where the line representing the severity of the symptom has been shifted along the when-axis.

### DETAILED DESCRIPTION OF THE INVENTION

The present inventors have devised ways to track an individual's food consumption and effects associated there with. Tracking consumption includes tracking the food items consumed and may include tracking the ingredients, additives, and nutrients associated with each food item (i.e. the constituents of the food). Effects associated with the consumption include changes in appetite associated with changes in metabolic rate and or consuming appetite inducing foods as well as any other physical, physiological, and psychological effects resulting from consumption of the food items. The inventors propose to use this tracking to better control one's diet as well as to determine any associations between food consumed and effects (symptoms) felt by the individual.

In an exemplary embodiment the inventors have designed a lifestyle regimen that includes traditional weight management and traditional deviations from the weight management norms, but in a new method that operates contrary to prior regimens. In particular, an exemplary embodiment of the innovative method disclosed herein prevents certain weight loss that may naturally occur when adhering to a weight loss regimen. The exemplary embodiment's approach attempts to reflect a holistic approach to weight management and in doing so it makes greater allowances for psychological aspects that influence success in dieting. Some of the psychological factors that heavily influence the regimen are those related to diet burnout. The principles underlying the exemplary embodiment of the method recognize that while some people consume too much of certain food items for simple reasons related to flavor etc, some people may do so for reasons that extend beyond just the flavor. For example, in some cases the dieter may associate a part of their identity, social or psychological etc, with the food items that are negatively influencing their weight and overall health. In such cases, a dieter may feel a certain loss of identity when adhering to a diet. Needing to retain/recover their identity may decrease the chances of success and lead to diet burnout. In other instances there may be a compulsive personality that drives the unhealthy consumption, and this same compulsive personality may be redirected toward a new diet, causing a heightened level of intensity for the diet, which may also eventually lead to burnout.

The method is further based on the recognition that a diet alone is unlikely to lead to long term health. The key to success is incorporating a diet into a healthy lifestyle, and this may best be accomplished by learning the principles underlying the weight management regimen. Consequently, part of the method disclosed herein includes educating the dieter. However, unlike prior techniques, the education may be delayed until after the start of the diet. This allows the dieter to see results first without having to undertake the daunting task of learning the underlying principles. The success is expected to then motivate the dieter to learn why they are succeeding and how they can further succeed.

In an exemplary embodiment one may first determine a person's starting body fat percentage and a desired body fat percentage, where the difference represents an amount of body fat to be lost. The body fat percentage can be determined using a body composition device ranging from a simple calipers operated by a person skilled in the measurement of body composition, to an electronic scale that sends an electric signal into the person and estimates body composition, to a hydrostatic tank operated by a person in the measurement of body composition. Alternately one may first determine the person's starting body weight using a body weight device such as a simple scale.

A target caloric intake is then determined for a period and is based on a target offset, which is a difference between energy spend and energy consumed as measured in calories. As used herein in the nutritional context, a calorie is the same as a Calorie, which is the same as a kilocalorie. The target caloric intake differs from the prior art. In particular, in the prior art there was an allowance for a maximum number of calories, but no caloric intake minimum was specified. A caloric intake minimum that would prevent a rate of weight loss acceptable for the diet, but faster than a rate of weight loss associated with the maximum caloric intake, would particularly not occur in the prior art, since such a caloric intake minimum would contradict the goal of the diet itself. However, in the lifestyle regimen disclosed herein the diet generates guidelines for nutrition content, and ways to determine a target caloric intake. In an exemplary embodiment the target caloric intake is determined based on the diet's maximum caloric intake as well as the other holistic factors, including, for example, the participant's level of confidence and/or commitment etc.

Part of the philosophy is that the target caloric intake is not only a maximum, but in a manner of speaking it is a minimum. It is hoped that the person employing the method disclosed herein will consume the target caloric intake; not consume more or less than the target caloric intake. It is understood that the person will not likely be able to hit the exact target caloric intake, so getting close is the emphasis. Hence, it is a target, not a minimum or a maximum. The target caloric intake can be determined simply by selecting a maximum caloric intake associated with a particular diet. Alternately it can be calculated in any of several ways. In a first way a target date when the subject on the diet wishes to reach the desired body fat can be selected, a rate of weight loss determined, and a target caloric intake necessary to reach that rate of weight loss calculated. In a second way the subject may simply set a target caloric intake at which they are comfortable. In this case the date at which the desired body fat percentage will be reached can be calculated.

It is known that each person has a base metabolic rate (BMR), which is essentially an amount of energy, in calories, expended by the individual at rest. A subject's total daily energy expenditure (TDEE) includes the BMR plus the day's exertion. In general terms, if one consumes more total calories in a day than the TDEE, one has a positive net caloric intake for the day, and a positive net caloric intake leads to weight gain. If one consumes the same amount of calories in a day the net caloric intake is neutral, and this leads to no weight gain. If one consumes fewer calorie in a day than the TDEE then one has a negative caloric intake, and this leads to weight loss. Whether one gains or loses weight depends generally on how these days cumulate over a period of time. Weight loss diets therefore will have a maximum caloric intake below the TDEE, but how far below the TDEE is often up to the person on the diet and can be limited by other health consideration.

It is also generally understood that diets fall into at least one of a few categories. A first category is a steady weight loss category and a second is a rapid weight loss category. Losing up to 1% of one's body weight, or approximately 1.5 to 2 pounds per week, (after the first week) is considered steady weight loss. Losing over this amount is considered rapid weight loss that may be adverse to the dieter's health. Consequently, when rapid weight loss is desired it is advisable to do it under a doctor's supervision. In either case, if the maximum caloric intake is to be calculated, it is a fairly straightforward calculation when the TDEE is known, the rate of weight loss desired is known, and the number of calories it takes to burn a pound of body fat is known. In addition, the person may select a diet where there is only a maximum caloric intake restriction. Alternately, the person may select a diet prescribed by a physician etc and which may include various other restrictions, such as maximums and/or minimums of certain ingredients, additives, and nutrients etc. The person may pre-select the diet, or may select a diet from a selection of available diets.

Once the target caloric intake is determined and the weight management program started, one then may monitor the person's actual caloric intake, which will be changed to be responsive to the target caloric intake. If the actual caloric intake equals the target caloric intake, then no caloric intake deficit (or surplus) is created. If the actual caloric intake is below the target caloric intake, then a caloric deficit is created. A caloric deficit, as used herein, is a difference between the target caloric intake and the actual caloric intake, if one exists. If a caloric deficit exists a person has not consumed as many calories as the regimen permits. If the person consumes less than the target caloric intake then a caloric shortfall is created. On any other diet, so long as it is not extreme, a caloric shortfall is generally regarded as a positive thing because it leads to weight loss at a greater rate than originally planned. In contrast, the method disclosed herein seeks to reduce or eliminate a caloric shortfall with a caloric intake up-adjustment. In stark contrast to the prior art, the method disclosed herein will prevent a rate of weight loss above the intended rate of weight loss, even if both are in the steady weight loss category.

While it is recognized that the caloric intake up-adjustment will prevent the possibility of weight loss at a greater rate than originally planned, the regimen views the caloric intake up-adjustment as an opportunity to prevent or forestall diet burnout. The regimen is willing to forego the potential for weight loss at a greater rate than originally planned for the opportunity to prevent burnout. It is believed that in the long run the person will better adhere to the regimen because it includes the caloric intake up-adjustments. As a result, burnout will be prevented or delayed, and in the end the person will lose more weight. Beneficially, the longer time on the regimen provides greater opportunity to educate the person about healthy eating and lifestyle, and it is believed that this will lead to a lifetime of better health.

The regimen differs from diets with spike days or cheat days. In those diets there is no mechanism for monitoring caloric intake shortfalls, as opposed to monitoring caloric deficits. In a spike day diet the maximum caloric intake varies from day to day, but remains the same from cycle to cycle of the diet. If one on the diet has a caloric intake shortfall, it remains. In cheat day diets there is virtually no monitoring of the cheat day intake; it is essentially ignored, and there is no correlation between the cheat day intake and the target caloric intake; whatever happens, happens.

The caloric intake up-adjustment can occur periodically, or it can occur when a certain threshold level of caloric intake shortfall has occurred. In an exemplary embodiment not meant to be limiting, one could recommend an up-adjustment weekly, for example, on a Saturday. (However, the up-adjustment could span more than one day.) This can be seen in FIG. 1, which shows a flow chart of the method and the up-adjustment process. The process 100 includes the steps of selecting at least one favored food 102, determining a target caloric intake for a period 104, determining an amount of calories desired for a caloric up-adjustment 106, and recommending favored food having a total caloric impact that best matches the desired amount of calories for the up-adjustment 108.

As used herein, a person's total caloric expenditure for a period, for example one day, is the total of their base metabolic rate, their daily non-exercise activity, and their exercise activity (a.k.a. TDEE—total daily energy expenditure). A target caloric intake is an amount of calories selected by the person to be consumed in a period in order to accomplish a particular weight management goal, such as a particular rate of weight loss or gain. If the target caloric intake is selected to be less than the total caloric expenditure, then a caloric deficit is intended. If the target caloric intake is selected to be more than the total caloric expenditure, then a caloric surplus is intended. A difference between the person's total caloric expenditure and the target caloric intake is an offset. The person may select a target caloric intake based on a weight management goal. For example, to lose weight at a certain rate of weight loss the person may select a particular target caloric intake that is below their total caloric expenditure. This is a negative offset. Conversely, to gain weight, the person may select a particular target caloric intake that is above their total caloric expenditure. This is a positive offset. An actual total caloric intake for a period is the actual amount of calories consumed by the person for the period. If the actual total caloric intake is less than the total caloric expenditure a caloric deficit is actually created. If the actual total caloric intake is greater than the total caloric expenditure a caloric surplus is actually created. If the actual total caloric intake for the period falls short of the target caloric intake then a caloric shortfall is created. Note that the caloric shortfall occurs when fewer calories than intended are consumed, as opposed to fewer calories than are burned. If the actual total caloric intake for the period is more than the target caloric intake then a caloric overage is created. An up-adjustment, alternately known as a correction, is meant to reduce or, ideally, eliminate any caloric shortfall. A deficit can be measured for a period such as a day or a week. A cumulative caloric deficit can also be measured where it spans several periods, such as a week when the cumulative caloric deficit is a day, or a month when the caloric deficit is a week. Likewise a cumulative caloric shortfall/overage can be measured where it spans several periods, such as a week when the caloric shortfall/overage is a day, or a month when the caloric overage/shortfall is a week.

The caloric up-adjustment seeks to reduce or eliminate the caloric intake shortfall from the target caloric intake for the period selected. For example, if during a week a person's TDEE is 2200 calorie per day, and if the person has selected a target daily caloric intake of 1700 calories, the result should be a caloric deficit of 500 calories per day. If, however, the person has consumed an average of 1400 calories per day over a time period of, for example, six days, from Sunday to Friday, the person has fallen short each day of a daily target caloric intake of 1400 calorie by 100 calories. If the up-adjustment were to occur on Saturday, the 100 calorie shortfall of each day would have accumulated to a 600 calorie shortfall for the time period of the week. Thus, any up-adjustment would seek to eliminate the 600 calorie accumulated shortfall by recommending food having a caloric impact of 600 calories.

Eliminating the caloric intake shortfall exactly, although ideal, is unlikely, due to the difficulty in finding just the right food item and/or number of servings to eliminate the caloric intake shortfall. Consequently, reducing the caloric intake shortfall is the more likely scenario. Reducing the caloric intake shortfall can be done by consuming any number of calories. The subject on the diet may reduce their intake shortfall from, for example, 600 calories to 50 calories below the target caloric intake. If they reduce it to 0 calories they have eliminated the caloric intake shortfall. If they had a caloric intake shortfall of 600 and consumed 700 calories, they reduced their caloric intake shortfall. In that case they also consumed 100 calories more than the target caloric intake for the period, but that may happen periodically, when the method considers all the factors, such as what favored foods are available, and what other considerations may come into play. Consequently, a reduction in the caloric intake shortfall may result in an actual caloric intake below, equal to, or above the target caloric intake for the period.

In an exemplary embodiment, a desired number of calories for the up-adjustment may be chosen. In an exemplary embodiment where the recommended food is selected solely from previously selected favored foods, it is unlikely that a single food or combination of food can be selected that generates a total caloric impact that is exactly equal to the desired number of up-adjustment calories. For example, if the desired number of up-adjustment calories is 480, chocolate brownies is the only favored food selected, and chocolate brownies have 200 calories each, then if the recommendation includes two servings of chocolate brownies the total impact may be 400 calories, which is 80 calories short of the desired 480 calories. If the recommendation includes three servings the total impact may be 600 calories and hence, 120 calories above the desired 480 calories. In this instance the best match may selected, where the best match is the recommendation that gets closest to the desired 480 calories. In this case since recommending two brownies gets within 80 calories of the desired 480 calories, and since recommending three brownies overshoots the desired 480 calories by 120 calories, the recommendation would be two brownies because it better matches the desired 480 calories.

In instances where multiple favored foods have been preselected, multiple combinations can be generated, each having a respective caloric impact. For example, if brownies and chocolate milk are selected, and the chocolate milk has 150 calories, then the recommendation would be a combination of two servings of chocolate milk having a total caloric impact of 300 calories, together with one serving of a brownie having a total caloric impact of 200 calories, for a total caloric impact of 500 calories. This combination of favored foods is the best match for the desired 480 calories because it totals 500 calories, which is only 20 calories above and is closer than any other combination will be when only favored foods can be selected for the recommendation. The food items consumed during the up-adjust may conform to the diet, or they may be contra-indicated by the diet. For example, most diets would not recommend an empty calorie food be consumed during the diet. This is usually based on a need to maximize food intake for the given calories to prevent excessive hunger pangs, as well as for maximizing nutrient intake. Similarly, relatively carbohydrate-rich foods such as potato chips or donuts would be contraindicated by a low carbohydrate and a ketogenic diet. Since eliminating the caloric intake shortfall will only return the subject's actual intake to the target intake of the regimen, and thus the intended rate of weight loss would remain intact, and since the up-adjustment is in addition to the person's target caloric intake for the day, increased hunger pangs will not be a problem, and hence almost any food item could be consumed at the discretion of the person on the diet.

In an exemplary embodiment the person may preselect one of more favored food items. These food items would be food items the person would prefer to consume during a caloric up-adjustment. The process may monitor the actual caloric intake and/or predicted actual caloric intake and based on an actual or predicted caloric intake shortfall, may recommend a food item or items from the list of favored foods. For example, if the person has a pension for chocolate chip cookies and has selected chocolate chip cookies as their favored food, the method may recommend however many servings of chocolate chip cookies, each with an associated caloric content, the person can consume to reduce or eliminate the caloric intake shortfall. In another example, if the person has an insatiable craving for beer and wings, and beer and wings are selected as favored foods, the method may recommend an allowable number of beer and/or wings that may be consumed to eliminate the caloric intake shortfall. The method may also recommend several options of combination of the favored food items, and the subject may select one of the options. Many parameters could be associated with an algorithm that makes the recommendation(s). For example, as in the latter example, the subject could limit a maximum number of wings desired, and request the remainder of the intake shortfall be eliminated with beer etc. However, any food can be consumed during an up-adjustment, not just preselected favored foods or foods contraindicated by the weight management program.

It is believed by some that a person's metabolism may adapt over time to caloric intake levels. For example, if one consumes fewer calories than one expends at a steady rate over a period of time the metabolism may adapt by slowing down. Alternately, under certain circumstance such as after periods of time of relatively low caloric intake, if one increases their caloric consumption the metabolism may adapt by increasing. This is one of the fundamental underpinnings of the zigzag type diets. How quickly the metabolism may adapt does not appear to be definitively known, and it may vary from one person to the next, but what is known is acceptable for the purposes addressed here. While some would say the adaptation may take weeks or more, others believe the adaptation can occur much more quickly, for example, in response to a variation in behavior that occurs during a single day. The variation in the metabolism may be less when the variation in behavior is relatively short etc.

One's appetite is often indirectly related to one's metabolism, and more particularly, to a size of a caloric offset (amount of deficit or surplus). An individual who attempts to maintain a deficit from day to day will experience a certain magnitude of deficit related angst otherwise known as hunger. For example, if one maintains a 500 calories deficit from day to day, one may experience hunger angst of approximately 5 on a scale of 1-10. The angst of 5 can be considered a satiety level of 5. Likewise, if the deficit were greater the angst would generally be greater. Since most people cannot obtain an exact number of calories they expend in a day they plan their consumption and deficit based on estimates of their caloric expenditure. Generally, if one's activity level for the day remains the same from day to day, the estimate for the daily caloric expenditure remains the same. However, it is believed by some that one's metabolism, and hence the calories one burns, may vary with the amount of calories burned via adaptation of the metabolism in response to the varying caloric intake. Consequently, these estimates of calories expended may not be entirely accurate.

If one estimates that they regularly expend approximately 2500 calories in a day, and the person wishes to maintain a 500 calorie a day deficit the person may consume 2000 calories per day. This may represent a relatively low caloric intake with respect to the relatively high caloric intake that occurs during the day of the up-adjustment. As such, the person may suffer with diet related angst of 5 out of 10. The inventors have recognized that, for a person whose metabolism does react relatively quickly, in the days after higher than normal caloric intake, such as a cheat day, the person's metabolism may adapt by increasing. However, when adhering to a calorie control regimen using most estimates the person would not adapt for this change. Consequently, the person conventionally would return to consuming 2000 calories to maintain their 500 calorie deficit. In reality the increased metabolism may increase the person's caloric expenditure by a transiently induced amount, for example, 200 calories. Thus, the inventors have proposed that on the day after a relatively high caloric intake, the person's caloric expenditure may be, for example, 2700 calories as opposed to 2500 calories. In this case the person who returns to their 2000 calorie intake would actually create a 700 calorie deficit. Since appetite is associated with a magnitude of the deficit, the person may suffer from increased hunger pangs, for example a 6 or 7 out of 10. While this may increase the rate of weight loss, one of the philosophies of this method is long term success. Increased hunger pangs are not necessarily conducive to long term success. While this explanation is thought to be a reason for an observed increase in appetite following a relatively large caloric intake, the inventors do not wish to be bound to this theory or the example magnitude of impact of the effect provided. Whether the cause of any subsequent increase in appetite is caused by this exact mechanism or another does not limit the method disclosed herein.

As a result, for those whose metabolism is thought to increase in response to a transient increase in caloric intake, the method innovatively seeks to prevent the increased hunger pangs by accounting for this transient increase in appetite induced by the sudden jump in caloric intake brought about by the up-adjustment during a short period following the up-adjusting consumption during which the metabolism is effected by the up-adjusting consumption. This may be accomplished by accounting for a change to the base metabolic rate (BMR) in a transient period during and/or following the increased consumption. The amount of increase may consider factors such as how many more calories were consumed than had been consumed leading up to the up-adjustment, how much of an offset there had been leading up to the consumption, and the person's sensitivity to changes in the BMR etc, and the changes may be linear or non-linear. For example, if the individual has a average actual daily caloric intake of 1600 calories, has a caloric shortfall of 1000 to correct, and on any other day would have a steady state target intake of 2100, then today's transient target intake could be 3100 calories. Consuming 3100 calories today would eliminate the shortfall and that would bring their average daily intake to equal with their steady state target daily intake. Stated another way, their average actual offset would then equal their target offset. Since their target offset is set based on their weight management goals, they would then be returned to their plan. However, their metabolism has become used to an average intake of 1600 calories and is shocked with a 3100 calorie intake on the up-adjustment day. This represents a caloric shock of 1500 calories, which nearly doubles what the metabolism has become accustomed to.

The metabolism may then become revitalized as a result of this caloric shock, and increase from whatever steady state value it had settled into. The amount of increase may consider the amount of caloric shock etc, as noted above. After the up-adjustment consumption the person returns to their diet plan and reduced caloric intake, so the metabolism will again settle back into an associated steady-state rate. However, this will not happen immediately, but instead may take one or more days. Accordingly, in the day or days during and/or subsequent to the up-adjustment, the BMR may be increased by respective amounts. For example, on a first day after the consumption the transient BMR for the day may be the steady state BMR increased by a first percentage. On the second day after the consumption the transient BMR for the day may be the steady state BMR increased by a second percentage. The second percentage may be less than the first percentage, or it may not. More or fewer days may be used.

On the first day following the up-adjustment in the example the BMR may be increased by, for example, 200 calories. Thus, the person would be permitted to consume 200 more calories because the target caloric intake is based on the BMR and the target offset. Since the BMR and the target intake are both increased by the same amount the person would maintain the target offset and would not experience any increased hunger pangs on the first day. On the second day the BMR may be increased by, for example, 100 calories and the target intake would be increased by 100. Here again, the target offset is maintained and hunger pangs are not increased. On a third day it may be modeled that the BMR has returned to steady state and so the target intake returns to its steady state value.

Other weight management plans have used caloric spikes to increase the BMR, but none have accounted for this increase when determining subsequent target intakes in an effort to reach a particular average offset and associated rate of weight loss.

It is also known that certain foods have secondary effects beyond the caloric impact on the diet. Certain foods, including but not limited to, sugar, high fructose corn syrup, monosodium glutamate, aspartame, refined mineral depleted salt, milk, processed foods, wheat (gluten), and alcohol actually increase appetite in a transient manner with little regard the person's total caloric intake. For example, if one eats 2000 calories of very high carbohydrate foods one may experience an increase in appetite that one would not experience if 2000 calories of, for example, pure protein were consumed. The food itself induces a chemical change in hunger response mechanism.

As detailed above, an individual's hunger response generates hunger pangs associated with a magnitude of caloric deficit or surplus (offset). For example, a 500 calorie deficit may cause hunger pangs on the order of 5 out of 10, and a 700 calorie deficit may cause hunger pangs on the order of 7 out of 10. However, this may be only one hunger response profile the body operates under. It is known that appetite is associated with an amount of offset, but it is also known that it is not directly proportional. An extreme example of this is a loss of appetite when one is ill. Despite being in a negative offset, one may experience reduced hunger pangs. Thus, when ill, when experiencing a negative offset (a caloric deficit) of 500 calories, the person may only experience hunger pangs on the order of 2 out of 10. Likewise, a negative offset of 700 calories may only elicit hunger pangs on the order of 3 out of 10. This change in hunger response demonstrates that there are intervening mechanisms that influence the hunger response. Consequently, one's hunger response can be modeled as profiles associated with hunger response parameters, and thus one experiences varying amounts of hunger for a given offset, and the amount of hunger depends on a state of the hunger response parameters.

As used herein the cause of these changes in the hunger response profile are termed chemical. Another example of a chemical change in the hunger response profile includes an increase in appetite when certain foods are consumed, such as those listed above that cause the transient increase in appetite. This may occur during the short period following the up-adjusting consumption during which the hunger response mechanism is affected by the up-adjusting consumption. For example, in some people consuming additional carbohydrates actually induces an increase in appetite. Thus, at a negative offset of 500 calories and a hunger response profile that yielded hunger pangs of 5, if one were to consume an unusually high amount of carbohydrates, subsequent to the consumption the person's hunger response profile may be chemically changed and the person may experience hunger pangs of 7 while at the negative offset of 500 calories. The carbohydrates themselves caused an increase in appetite. Another example of this chemical change is an increase in appetite that invariably results from consuming alcohol.

Which hunger response profile a person is experiencing is determined by the hunger response parameters. While it is likely impossible to ascertain every parameter involved, it is believed that the parameters include a composition of food that the individual has been consuming for a period of time. For example, if the person is on a diet that prefers a certain composition of food be consumed, the person's hunger response profile will settle into that diet composition when the person spends a sufficient amount of time on the diet. While it may change from one individual to the next, it is believed that a person will enter a steady state hunger profile after several days on a particular diet composition. For example, certain low carbohydrate diets tend to require 4-6 days until the body adjusts. Likewise, if the person is on a ketogenic diet etc for a sufficient period of time they will likely settle into an associated hunger response profile. (Weight loss and weight gain also have commonly accepted ratios of proteins, fats, and carbohydrates as well as recommended daily allowances for nutrients etc, and these would cause the formation of an associated hunger response profile.)

The associated hunger response profiles may or may not be the same as each other. However, what matters is the change that occurs from whatever hunger response profile they are experiencing as a result of consuming foods. Consuming a relatively high number of appetite-inducing carbohydrates during an up-adjustment while on a low-carbohydrate diet will undoubtedly cause a relatively large transient change in the hunger response profile in a carbohydrate sensitive person, and hence an allotment in the recommendation would be greater under this scenario. (Conversely, when on a relatively high carbohydrate, weight-gain diet the allotment may be lower due to the ubiquitous presence of carbohydrates recently consumed). Often, it is exactly this type of scenario that is encountered for one on a diet, where the desired food is of a composition so different from that of their regular diet, or even contra-indicated by their diet, that the food itself induces the chemical change in the hunger response profile.

This chemical change in the hunger response can be devastating to a diet. For example, for one who has reached a steady state hunger profile while on a diet of a particular composition, that one may experience hunger pangs of 5 while at a given negative offset. After consuming the appetite-inducing foods the person may subsequently experience hunger pangs of 7 at the same negative offset. To reduce their hunger angst from 7 back to 5 the person is likely to consume more food. This reduces the negative offset which, in turn, reduces the rate of weight loss. Thus, the up-adjustment has a greater impact than simply that of a one-time increase in caloric intake. It changes the person's hunger response and the change cases additional increases in caloric intake. This cycle can continue and can feed upon itself and in certain cases could throw the person into a caloric surplus, thereby devastating the diet.

The present inventors have recognized this chemically induced subsequent increase in appetite and instead of ignoring it like has traditionally been done, have devised a way to account for it in the up-adjustment. The method accounts for the induced increase in appetite and incorporates it into the up-adjustment so that the caloric value of the recommended food plus the induced and subsequent increase in consumption necessary to satiate the transient increase in appetite together reduce or eliminate the shortfall. Since the increase in appetite is transient because the person returns to consuming foods associated with their selected diet after the up-adjustment, and hence returns to the associated hunger response profile, the recommendation reduces or eliminates the shortfall without deleterious effects of induced appetite. When used together with the techniques described above for adjusting intake based on a change in metabolic rate, the inventors are able to reduce or eliminate the shortfall without deleterious effects of either induced appetite or increased metabolic rate. This brings the person's average daily offset closer to (or matches it with) their target daily offset, and this brings them closer to (or matches) their intended rate of weight loss. This permits them to enjoy a periodic splurge without fear of the aftermath, which helps morale and consequently helps them adhere to their diet plan.

This transient increase may span a time span less than a day or may span one, or even more than one day. In an exemplary embodiment the method may look at the ingredients, additives, and nutrients of the favored foods. If an appetite inducing food is present, a hunger response allotment for the food-based increase in appetite is estimated, which is an amount of calories that will be necessary to compensate for the increased appetite. This hunger response allotment is included in the total caloric impact of the recommended foods. The caloric impact, as used herein, includes the caloric content assigned to the food, but also includes the hunger response allotment necessary to satiate an expected transient increase in appetite brought about by the food. The hunger response allotment can be made in amounts proportional to the amount of each appetite-inducing constituent/consumable. For example, if a food item is consumed and it has both alcohol and salt, such as a margarita, then the hunger response allotment of the margarita may include a factor for the caloric value of the margarita itself, a factor for the alcohol in a proportion associated with the amount of alcohol present, and a factor for the salt in a proportion associated with the amount of salt present.

For example, if the induced appetite caused by consuming food item 1 could be allayed by consuming another 50 calories of food in the ensuing time, then one serving of favored food item 1 would have an associated food-induced value of 50 calories. Since favored food item 1 has, for example, 110 calories, and induces an increased appetite that takes 50 calories to satiate, the method may include the 50 calorie allotment as part of the up-adjustment. For example, if a caloric intake shortfall of 330 calories exists, in one exemplary embodiment that does not consider the induced appetite the method might simply recommend 3 servings of favored food item 1, which would eliminate the caloric intake shortfall completely. In another exemplary embodiment that does account for the induced appetite, the method may only recommend two servings of favored food item 1, because it also knows that the two servings will induce an increased appetite in the person, and it will take 50 calories for each serving of favored food item 1 to satiate the increased appetite. Thus, the two servings of favored food item 1 would be 220 calories, and the 100 calories (2 servings @ 50 calories) of increased consumption would add up to 320 calories, and this would almost entirely eliminate the caloric intake shortfall of 330 calories.

It is understood that determining an exact amount of induced appetite any given favored food item induces may be difficult to quantify, and the exact amount may not be linear with the number of servings. It is also understood that it may be difficult to quantify the exact amount of calories it may take to satisfy the induced appetite. It is still further understood that these values may vary from person to person, since each person's physiology varies. As a result, the up-adjustment may initially use estimates for the amount of induced appetite and the amount of calories needed to satiate the induced appetite arising from consuming a given favored food item. In time the person on the diet could adjust up or down the estimates based on experience with the initial estimates. However, even if only using initial estimates, the method still provides an up-adjustment that protects the person on the diet from the effects of the recommended food. No longer is the person left to suffer with an increased appetite resulting from their cheat day. Instead, the holistic approach allows the person to further enjoy the up-adjustment, knowing full well that they will not have to suffer the next day for their actions that day.

In light of the above, it can be appreciated that the total caloric impact of the food in the up-adjustment can include 1) an allotment for the caloric content of the food included in the recommendation, and 2) a total allotment for the transient increase in appetite brought about by consuming certain foods, where the total allotment may have several components, where each component may be associated with each appetite-stimulating constituent present in the food item. One, the other, or both of these factors may be used as necessary to reach a proper total caloric impact for the up-adjustment. Thus, using the example from above, if the person with an average total caloric expenditure of 2500 calories has a cumulative 800 calorie shortfall to be eliminated, the up-adjustment total caloric impact should be 800 calories. If, for example, the person wishes to consume their beloved beer and chicken wings as the up-adjustment, the up-adjustment may include a combination of beer and wings where the caloric content of the food is 500 calories, an allotment for the increased appetite due to consuming alcohol, which is known to induce appetite, may be 200 calories, an allotment for the carbohydrates in the beer, which are known to induce appetite, which may be 65 calories, and an allotment for the sodium in the Buffalo wing sauce, which is also known to induce appetite, may be 35 calories. (These values as well as the number and type of appetite stimulating constituents/consumables given are purely for explanatory purposes; true values may be different and more or fewer constituents/consumables may actually exist in such combinations.) The reality of this induced appetite has been felt by many folks the day after a party, and many of those who expected to enjoy one cheat day from their diet have ended up losing two days or more of progress instead of one as a result of this induced appetite. The innovative method disclosed herein is an effort to account for the entire caloric impact of the up-adjustment, not just the face value of the caloric content, so that one can better control their intake and its effects, and thereby better adhere to their diet.

From the foregoing, it can be seen that an up-adjustment may include both transient modifications to a person's steady state BMR and target intakes that are necessary to bring the person's consumption in line with their chosen steady state offset. Prior to the consumption the method is capable of providing a recommendation that, if followed, will reduce or eliminate the person's shortfall while not causing them to experience any increased hunger pangs. This is substantially different from prior art techniques which do not account for the impact of the up-adjustment in their target intake values or the subsequent impact on the magnitude of the person's hunger pangs. The method can utilize transient values for the BMR, an allotment for the chemically induced increase in appetite, or both as is desired.

In another example, the person has an average TDEE of 2600 and a target offset of −500 (a 500 calorie deficit), which yields a target intake of 2100 calories. The person has accumulated a shortfall of about 1000 calories. This may happen if, for example, the person only consumes 1900 calories for the most recent five days. {((2100−1900)*5)=1000 shortfall}. The person has a pre-selected list of favored foods that includes chicken wings and beer, and the person wants to consume both during the up-adjustment. A recommendation of two beers and three wings may be presented. The caloric value of the food may be approximately 800 calories. This recommendation may be selected because it leaves a 200 calorie hunger response allotment. If the person consumes the recommended food the person may then experience an increase in appetite induced by the composition of the food, and this chemically induced increase may cause the person to consume 200 more calories subsequent to the consumption to reach the same level of hunger satiety that would have been achieved were non appetite inducing foods consumed. Consequently, the transient target intake may be increased by 200 calories. The 200 calories may be divided and added to the target intake for more than one day, or it may be applied in one day. Thus, assuming for the moment no change to the BMR, the transient target intake subsequent to the up-adjustment may be increased from the steady state of 2100 calories to 2300 calories. If there were no change to the BMR then the person is being directed to eat more than the steady state target intake, and hence have a reduced deficit for the day, but this is necessary to prevent increased hunger, and since it is allotted for, it will further reduce the shortfall without creating an overage, which is the goal.

However, since the person in this example actually follows the recommendation, the person consumes 2900 calories during the up-adjustment, {2100 TDEE+800 shortfall} so the person would experience a caloric shock 1000 calories {2900−(1900 for 5 days)/5 days}, so an increase to the BMR is calculated. If the increase (a.k.a. a BMR allotment) is calculated to be approximately 200 calories, then the person's TDEE, which includes the BMR, is also increased by 200 calories. Thus, subsequent to the up-adjustment, the person's TDEE may be the typical daily TDEE increased by 200, yielding 2800 calories for the TDEE on that day. The transient target intake would then be 2300 {2800 TDEE−500 offset)=2300}. However, since the person consumed only 800 calories, the unmet shortfall {1000 shortfall−800 consumed during the up-adjustment} is added to the target intake, yielding a transient target intake for the day of 2500 calories. Thus, in this example, subsequent to the up-adjustment the person's transient target intake is 400 calories above their 2100 calorie steady state target intake. This is a significant amount of consumption permitted only by this method and not permitted by prior art methods. Were these methods not being used and the person were returned to the steady state target intake of 2100 after the up-adjustment, the increase in metabolism would increase the person's actual deficit by 200 calories, and the chemical change to the appetite would make the person feel like they were experiencing 200 additional calories of deficit. The result under the conventional methodology is that the person would be experiencing hunger-related angst equivalent to that of a 900 calorie deficit as a result of the up-adjustment. However, using the techniques described herein, the person not only eliminates their shortfall; they do so without experiencing any increase in hunger pangs. Their negative offset for the day would be 300 calories {2800 TDEE−2500 transient target intake}. Since their hunger response profile has temporarily changed from a steady state response (associated with the composition of food associated with their chosen diet) to the transient hunger response profile where an increase in hunger is chemically induced (resulting from the increased consumption of the up-adjustment), at the 300 calorie deficit they will experience hunger pangs similar to what they would experience at a 500 calorie deficit under the steady state hunger response profile. Stated another way, in this example the person feels the same level of satiety during the period following the up-adjustment consumption where changes resulting from the consumption are in effect in the body as before the up-adjustment consumption despite changes to the BMR and/or the target offset, while in contrast, in the prior art methods the person would experience increased hunger pangs.

The methods discussed herein can be implemented without ever performing a recommendation. For example, if the person never requests a recommendation but instead indicates that they recently consumed more calories than their target intake, such as during the day before, changes to the BMR can be implemented for the instant day, and any unmet shortfall can be added to the transient target intake for the instant day, or the unmet shortfall can be spread out over more than one day by increasing the transient target intake by respective amounts that add up to the unmet shortfall. Similarly, if a recommendation is presented and the person instead chooses to reduce or eliminate the shortfall by consuming other foods, the TDEE after the up-adjustment as well as the transient target intake can be calculated based on what was actually eaten. When that which is actually eaten is the same as the recommendation, then the calculated figures will be the same as the predicted figures. If the person consumes a caloric surplus on the up adjustment day {actual intake—target intake} equal to or greater than their shortfall during the up-adjustment, then the BMR may be adjusted as necessary, but there would be no unmet shortfall to add to the transient target intake. Stated another way, if a caloric overage is created, there would be no subsequent increase in the target intake due to the chemically induced appetite because this would over-correct. In addition, the recommendation can be presented without following up on what is actually consumed. This may happen if the person initially requests a recommendation but then does not act to reduce their shortfall, but instead sticks to their regular diet.

In the scenario when the person initiates an up-adjustment but does not eat enough calories to overcome their previous shortfall, their running caloric shortfall can still be monitored through the up-adjustment period. However, the running shortfall should not double count the previous shortfall that may have been incorporated into the transient caloric goals. For example, if the person's steady state target offset is a 500 calorie deficit, but during the up adjustment period the transient target offset has been adjusted to a 300 calorie deficit in order to eliminate the previous shortfall, their shortfall for that period would be based on the steady state target offset of the 500 calorie deficit, not the transient 300 calorie deficit. This is because the transient target of a three hundred calorie deficit includes prior shortfall, while the steady state target offset of the 500 calorie deficit does not. To avoid double counting any prior shortfall that has been included in the transient target, the shortfall calculation for that period should be based on the steady state offset from the TDEE, which is then measured against the period's caloric intake. During the up-adjustment period the TDEE number may also change because the BMR changes, but the shortfall calculation does rely on the TDEE that occurs during the up-adjustment because the TDEE that occurs during the up-adjustment period is unrelated to (not based on) any prior shortfall and therefore no prior shortfall would be double counted.

While the examples given have focused on weight loss regimens, the same principles apply to weight gain regiments. For example, if the person wishes to maintain a certain positive offset (surplus), then any shortfall is corrected by similarly causing them to increase their consumption.

Several psychological advantages may be realized from the caloric up-adjustment. At a minimum the person on the diet will have something to look forward to. After several weeks of this it will also become apparent to the dieter than the weekend, and the favored foods, will always be there. They will understand that they need to refrain from excessive consumption, but they need not change their lifestyle entirely. This may help them feel they can retain a sense of identity throughout. This, in turn, eases the transition from their prior lifestyle to the new lifestyle. It is no longer old lifestyle versus new; it is simply a rebalancing of their lifestyle. This may alleviate any pride issues related to the choices made in the prior lifestyle, and increase acceptance of the healthier lifestyle they are embracing.

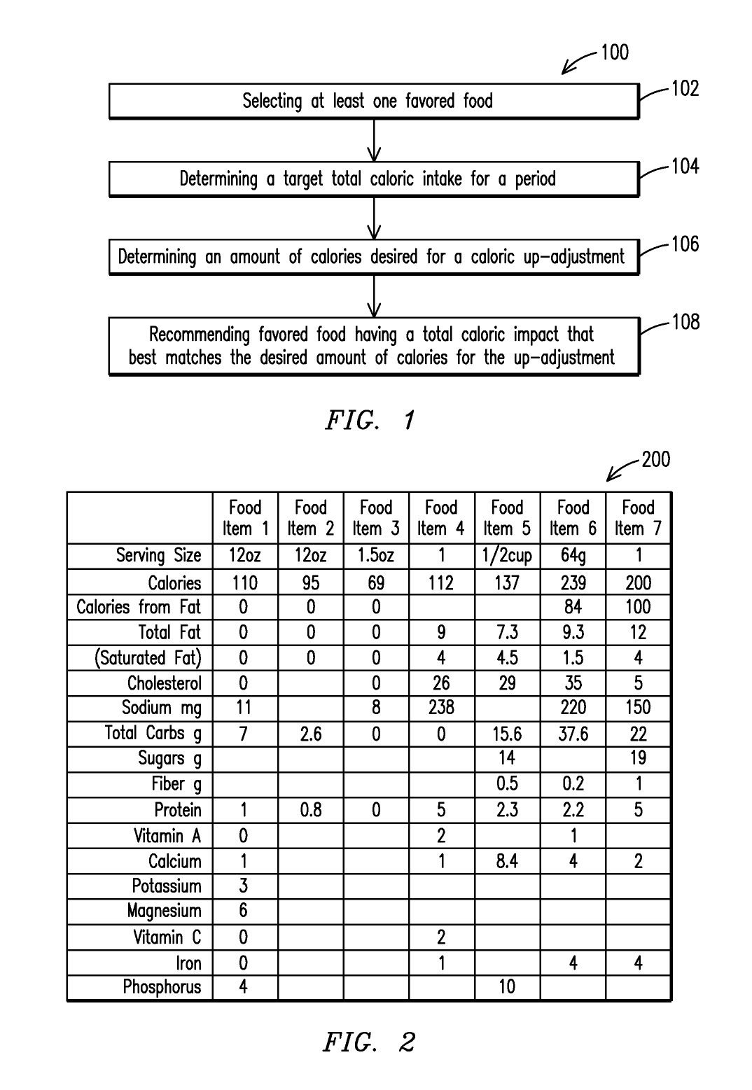

Heretofore the discussion has focused on calories. In instances when there is only one favored food, the recommendation is necessarily limited to a number of servings of the favored food. However, in instances where there are several favored foods selected, the method may focus more deeply on the favored foods in the recommendation. In particular, account may be taken of composition of the food, including the ingredient, additive, and nutrition content. For example, each favored food may have a different composition of fats and carbohydrates. This can be seen in FIG. 2, which is a chart 200 of various exemplary food items and associated nutrition content. It can be seen that food item 1 has 110 calories, but no fat. Food item 7 has 200 calories, and 12 grams of fat. In addition to monitoring a person's actual caloric intake, the method can monitor the nutrition content of the consumed food. This could be done by manually entering the nutrition value for each food item into a computer having software necessary to execute the method. Alternately, the person may enter the consumed food item in the computer and the software may access a database, such as a third party database that has nutrition information for a wide variety of food items, and retrieve the likely nutrition content information for the consumed foods.

It is generally known, for example, that a certain amount of fat is needed in a person's diet. The possibility also exists that the person on the diet may have specific requirements imposed by their particular diet. Using fat as the example nutrient, the diet can compare the person's actual fat intake and compare it to the person's preferred fat intake. If the person has a fat intake shortfall, the method may recommend food item 7 over food item 1 to reduce or eliminate the fat intake shortfall. This can be determined by calculating a projected amount of the nutrient that will be consumed if the food in the food recommendation is consumed. The actual intake up to that point together with the projected intake would be compared to the target intake for the nutrient.

Another example nutrient could be salt, and someone on a fat loss diet who also has a heart condition may have a further restriction regarding salt intake. In such a case the diet may avoid recommending food items 4 and 6 if a caloric intake shortfall exists. Alternately, a recommendation may be made where the calories are simply cut back to a point where the maximum level of the restricted nutrient, (or other ingredient or additive) is not exceeded. For example, if the person is on a restricted sodium diet but only has Buffalo wings as a favored food, a recommendation that could support three wings for a person not on a sodium restricted diet may be reduced to two wings for someone who is on a sodium restricted diet. This may occur if consuming three would cause the person to exceed their sodium intake for an allotment of time such as a day while consuming two would not. This can be determined by projecting the actual intake of the restricted constituent if the recommendation is consumed and determining if the projected actual intake would exceed the maximum level of the restricted constituent. Alternately, a recommendation may consider a constituent other than a restricted constituent. For example, if the person is not consuming enough carbohydrates, such as a person on a muscle-gain regimen, and if the favored foods include foods of varying carbohydrate content, the recommendation may select a food or combination that satisfies both a reduction in the caloric shortfall and a reduction in the carbohydrate shortfall. For example, if food combination 1 reduces the caloric shortfall by “X” but has little carbohydrates, while food combination 2 reduces the caloric shortfall less effectively but has twice the carbohydrates, food combination 1 may be selected if caloric shortfall reduction is preferred, while food combination 2 may be selected if carbohydrate shortfall reduction is preferred.

In some cases the recommendation may not be able to reduce or eliminate the caloric intake shortfall and a nutrient intake shortfall without exceeding one or both of the targets by some degree and this may be acceptable. For example, if it is deemed that reaching the preferred nutrient intake is more important than not exceeding the target caloric intake, the process may recommend a favored food item or collection of favored items that result in a best overall match. The best overall match can be determined in a variety of ways. For example, weights may be given to a value of the target caloric intake and to each of the nutrients, where the weight represents a level of importance to the subject on the diet. If several nutrients are being monitored, such as fat, total carbohydrates, cholesterol, sodium, and potassium, a best match, i.e. the most effective match, may be a combination of favored food items that first favors reducing or eliminating a respective intake shortfall/surplus for the weighted nutrients and then minimizes any respective intake shortfall/surplus for the lower weighted nutrients etc. Consequently, a reduction in a nutrient intake shortfall may result in a respective actual nutrient intake below, equal to, or above the respective preferred nutrient intake.

In another exemplary embodiment the process may perform the above steps, but instead of correcting any nutrient intake shortfall(s) as part of a caloric up-adjustment, the process may independently suggest food items from any number of other databases unrelated to the favored foods that conform to the diet and which reduce or eliminate a nutrient intake shortfall(s). For example, if a nutrient intake shortfall exists, the process may suggest a vegetable high in that nutrient. This can be executed entirely independent of the caloric up-adjustment.

In another exemplary embodiment the process may monitor the actual caloric intake and if there are any unanswered actual or predicted intake shortfalls and/or surpluses during the diet the process will adjust the date when the process predicts that the person will reach the target body fat percentage. The process can also include an exercise plan in the calculations. For example, if an exercise plan has the person expending a given number of calories, this number can be included in the TDEE and considered when determining the daily caloric deficit. Whatever exercise is actually done can be monitored and the target date can be adjusted based on the exercise performed.

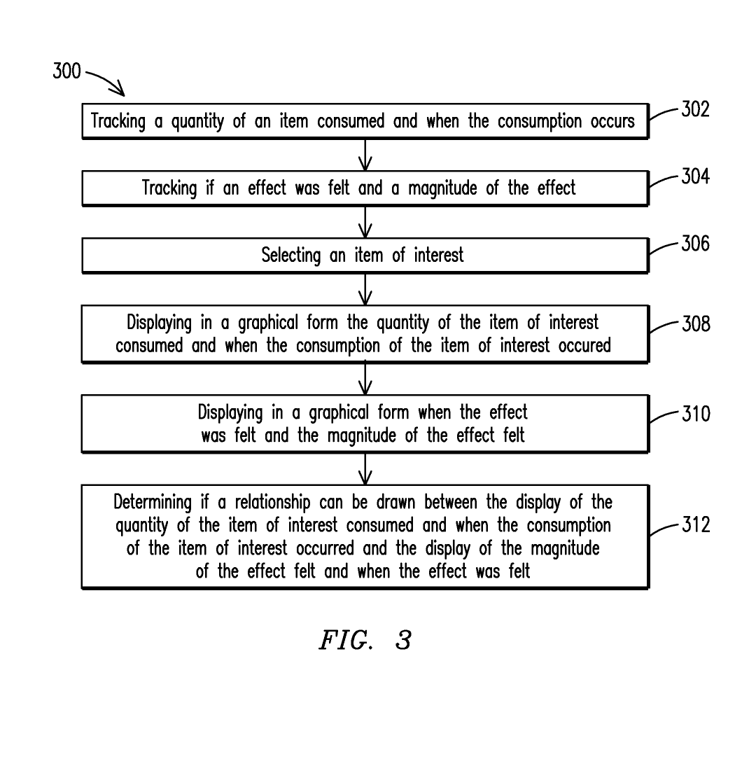

The process may also make information about the food item's ingredients, additives, and nutrients in the food available to the person in a manner that may allow the person to draw inferences about their health. This is described in FIG. 3, where the method 300 includes the steps of tracking a quantity of an item consumed and when the consumption occurs 302, tracking if an effect was felt and a magnitude of the effect, 304, selecting an item of interest 306, displaying in a graphical form the quantity of the item of interest consumed and when the consumption of the item of interest occurred 308, displaying in a graphical form when the effect was felt and the magnitude of the effect felt 310, and determining if a relationship can be drawn between the display of the quantity of the item of interest consumed and when the consumption of the item of interest occurred and the display of the magnitude of the effect felt and when the effect was felt 312.

Ingredient information, additives, and nutrients are commercially available through various database information vendors. For example, in an exemplary embodiment the food items actually consumed may be monitored and items of interest tracked. Food items as used herein includes essentially anything that a person eats, including groceries etc, medicine, health supplements/vitamins etc. Items of interest as used herein includes the food items itself and any ingredient, additive, or nutrient in consumed foods. This is so because in some cases an item of interest may be a food item itself and an ingredient within another food item. For example, one can consume sugar as part of an energy drink and one can consume sugar directly. Hence, the item of interest is in one instance the item consumed and in the second instance it is part of the item consumed. It is for this reason an item of interest is distinguished from how it is consumed. Thus, when sugar is an item of interest, it can be tracked if consumed as part of a food item and also if consumed directly in the same time period. The process may be configured to display the item(s) of interest in, for example but not meant to be limiting, chart form. Each item(s) of interest may be charted on its own chart and actual intake may be shown with preferred/recommended intake. Alternately, one or more item(s) of interest may be charted together on a single graph. The process may allow the person to track a severity of specific effects including physical, physiological, and/or psychological items in, for example, similar charts. Physical effects include pain etc. Physiological effects include illness, sleep, digestive tract ailments, blood pressure, etc. Psychological effects include mood, such as happiness, grumpiness, depression, unclear thinking etc. The effects may be entered as part of, for example, a log. For example, if a person is having problems with their skin, or mood swings, or internal distress, a severity/magnitude of the symptom can be logged. Then the actual intake of selected item of interest may be compared with the magnitude of the effect to see if a correlation can be derived between an actual intake of the item of interest and the magnitude of the effect. Effects need not be limited to negative effect/ailments, but may also include positive conditions, such as improvements over time. For example, if the person has stomach aches periodically, an actual intake of gluten can be monitored and compared with a severity of the stomach aches to see if gluten might be associated with and/or responsible for the stomach aches. Any number of items of interest can be monitored, and any effect can be tracked. Further, the process may seek to find relationships between severity of symptoms and any possible combination of items of interest that may be aggravating or aiding the symptom.

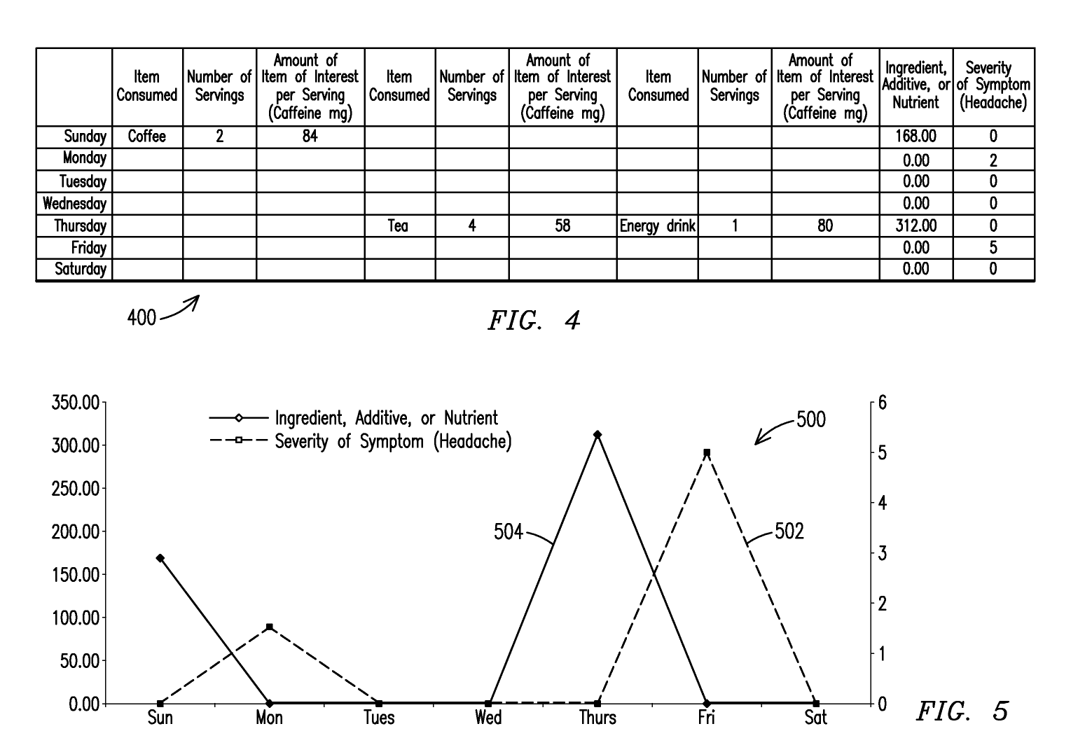

FIG. 4 shows an example of particular food items and an item of interest consumed over time. In an example using this information the person may periodically experience headaches and may not know what causes them. The person documents a severity of the headache and shows it occurring on Monday with a severity of two and on Friday with a severity of 5. Perhaps the person heard that coffee may cause headaches so he didn't drink coffee after Sunday. Perhaps this person substituted an energy drink for coffee on Thursday, and then later chose to have some soothing tea. If this person sought a relationship between the headaches and consumed food items only, such as coffee, one might not be found because there is no common food item consumed on the day of or reasonably before the headaches. However, the inventive method disclosed herein tracks constituents of consumed food, including caffeine. If the person were to graph caffeine intake and a severity of the symptom as shown in the FIG. 5 x-y graph 500, the person could see a relationship between a shape of the line graph 502 representing the severity of the headache and a shape of the line graph 504 representing the intake of caffeine. In this graph time is shown along a “when-axis”, the magnitude of the effect is indicated by a “magnitude axis”, and an amount of the intake is indicated by a “quantity axis.” This graph totals the intake of a single constituent that may have been present in several different food items consumed during a particular time period, such as a day, and graphs these time periods over a longer time, such as a week. Thus, the caffeine present in the energy drink consumed on Thursday was added to the caffeine present in the tea and the total caffeine consumed for the day was displayed on the graph along the total for other day for a period of a week. The innovative method of tracking the items of interest, grouping the items of interest together in a given time period, and graphically representing them together with an effect being investigated is hoped will provide insight and eventual relief for those seeking to find a food-induced cause of an effect they may be feeling.

Other examples where this tool may be found useful is in determining if a person has an allergy to a consumable. For example, if one suspects they are allergic to a particular constituent, they may approach it from the other direction by graphing their intake of the constituent and then searching through effects to find an unsuspected relationship. It is understood that as many as ninety percent of food-allergic reactions are linked to a handful of allergens, in particular: milk, eggs, peanuts, tree nuts (such as almonds, cashews, and walnuts), fish (such as bass, cod, and flounder), shellfish (such as crab, lobster, and shrimp), soy, and wheat. Other, rarer allergens include corn, meat, gelatin, seeds, spices, and certain medicines. Additionally, one may seek a relationship between an item of interest and a reaction associated with an intolerance, for example gluten intolerance. Still further, if two constituents work synergistically to cause an effect this can be discovered using the method and tools disclosed herein. While it may be harder to find a synergy, one may simply graph two items of interest on the same graph as a severity of an effect/symptom.

If a relationship is determined between an item of interest and a magnitude of a symptom the person may reduce an amount of the item of interest consumed in a subsequent time period, re-graph the graph of FIG. 5, and determine if a reduction in the intake of the item of interest actually resulted in a decrease in the magnitude of the effect felt. If so, that lends credibility to the conclusion that the item of interest is indeed causing and/or exacerbating the effect felt and the person can now learn to control the magnitude of the effect felt by controlling their consumption of the item of interest.

In yet another example, one may wish to determine a relationship between an item of interest and a physical effect. Perhaps the person is on a fitness regimen where muscle gain is a goal. The person wishes to determine what items of interest are most beneficial to their muscle gain. The person may wish to track their maximum performance over a period of time and compare a rate of improvement when consuming one item of interest to a rate of improvement when consuming another item of interest. If there is a noticeable difference in the rates of improvement the person may wish to consume the item of interest that provides the greater rate of improvement over the other item of interest. The person may wish to measure, for example, their maximum bench-press capacity. This can be a measure of a one-time maximum bench press, or alternately, it may be a measure of how many times the person can bench-press a given weight. In this example the person consumes a first item of interest conducive to muscle growth, such as a first type of protein, for a period of time sufficient to establish a comfortably accurate first rate of bench-press improvement (i.e. increased one-time maximum, or increased number of repetitions), such as four months. The person may then switch to a second type of protein and measure their second rate of bench-press improvement over a second sufficient time period. Upon completion the person may compare the first rate of bench-press improvement to the second rate of bench-press improvement to determine then better. From this the person may be able to conclude that the protein associated with the better rate of improvement is better for them. It is understood that there are multitudes of types of effects that can be investigated that extend well beyond those presented herein.

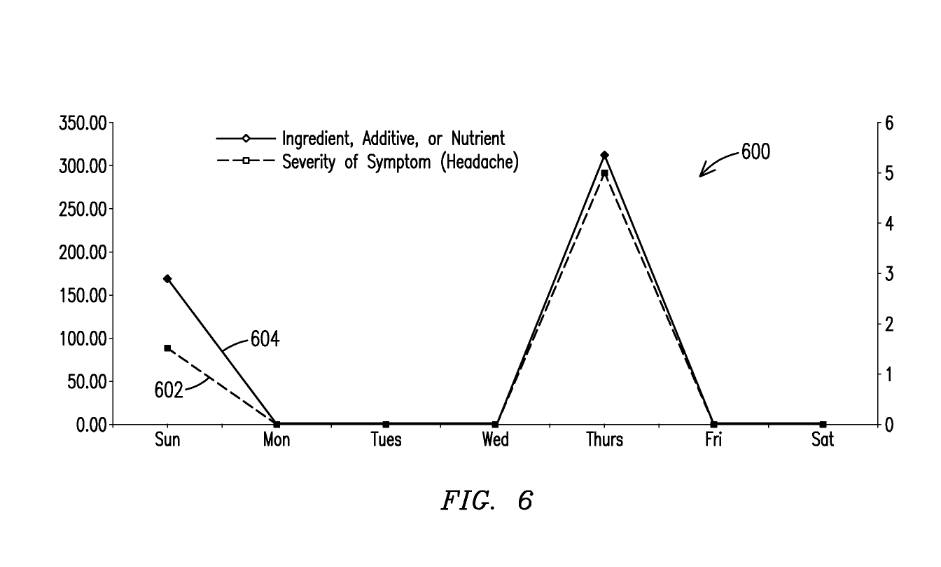

It is appreciated that the example given in FIG. 5 is very simple in nature. This is done so to illustrate the concept, but it is understood that the relationships may not be so clearly defined, and may not occur so close in time etc. For example, it may be that an effect caused by consuming an item of interest might not manifest until several days later. In this instance it may be difficult for a person to draw a correlation between the patterns of the line graphs representing the amount of consumption of the item of interest and the severity of the symptom. For this reason the inventors innovatively propose that one of the graphs be adjustable along the “when-axis.” This can be seen in the FIG. 6 graph 600, where the line representing the severity of the effect 602 has been shifted along the when-axis one time period (one day) to the left, while the line representing the intake of caffeine 604 has not been shifted. As a result of this the relationship between the patterns of the two lines is made more visually evident. If the cause and effect were spread over several days or a week any correlation between the patterns would made much easier to recognize. These charts can be physically displayed in any manner, including an electronic display screen, paper and pencil etc. Visually displaying these lines in this manner is extremely effective in helping one determine any relationship in the patterns that might not be evident by looking at numbers alone.

In an exemplary embodiment the process may include an educational component. Often, when undertaking a diet, the person may feel overwhelmed at the amount of learning necessary not only to know what is acceptable food, but how to prepare it, and how to prepare it in a manner that makes it seem enticing. Further, simple and pragmatic things, such as where to go to obtain the right food items and/or ingredients, may seem elusive. As a holistic approach, the process addresses these concerns. In an exemplary embodiment the regimen may have one or more core menus. A core menu may include recommended menu items that the person may eat for a period of time, for example, two weeks. With this core menu may be associated a core menu lesson plan. The core menu lesson plan may include information related to the menu items in the core menu. The person could stick with the core menu items indefinitely, but it is envisioned that the person may tire of these menu items. At this point the person is motivated to review and/or complete an expanded menu lesson. The expanded lesson plan includes information about menu items not included in the core menu. Once the expanded lesson plan is completed, the person will have acquired additional knowledge and will then be able to expand the menu items available to them as a reward for the progress. This can be repeated until the person has a working knowledge of what is acceptable and can formulate their own menu items.

Part of the education includes the semantics of where to purchase the healthier groceries necessary to prepare the menu items. This may be due in part to the fact that certain grocery items may not be available in the places known to the person. In an exemplary embodiment the process may generate a list of grocery items by accessing a database, such as an internal or third party database, where ingredients for food items are delineated. In another exemplary embodiment, the process may indicate where the grocery items are available for purchase by accessing an internal or third party database. In an exemplary embodiment the process may generate a route indicating the best choices to get the grocery items. The route may consider such factors as prices of the grocery items at differing stores, distance, congestion etc.

In another exemplary embodiment the process may include a personal visit to the person on the diet by an educator. The educator may guide the person on the diet through the person's food stores and indicate, verbally, or physically via markers or labels etc, whether the respective food/grocery item is good, marginal, or poor with respect to guidelines established by the diet the person selected.

In light of the foregoing, it is evident that by monitoring a person's intake in this particular way the present inventors may be able to prolong the duration one remains on a diet by considering factors beyond those limited to the diet and caloric intake. As a result, the process disclosed presents a unique and counter-intuitive way to increase someone's likelihood of success with their weight loss effort. Furthermore, the innovative monitoring enables individuals to investigate and possibly identify a food-related root-cause for any effects they may be feeling. Consequently, the matter disclosed herein represents an improvement in the art.

It should be understood that the system and process disclosed herein may be implemented in any appropriate operating system environment using any appropriate programming language or programming technique. The system can take the form of a hardware embodiment, a software embodiment or an embodiment containing both hardware and software elements. In one embodiment, the system is implemented in software (controls) and hardware (sensors), which includes but is not limited to firmware, resident software, microcode, etc. Furthermore, parts of the system can take the form of a computer program product accessible from a computer-usable or computer-readable medium providing program code for use by or in connection with a computer or any instruction execution system. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD. The display may be a tablet, flat panel display, PDA, or the like. For example, the process may be implemented through the internet. A user may input data at a remote terminal. As used herein the term remote means that some form of communication tool is necessary to implement the method because the components used to effect the method are located far enough apart from each other that direct human-to-human communication, such as voice, is not possible. For example, the data may be stored in a database at a remote location from the person seeking a recommendation or creating a chart for pattern comparison, and in this scenario the method cannot be performed solely in a person's mind. The data may be retrieved at a later time for analysis by a processor at yet another remote location. Finally, the graphs may be presented on an electronic display at any terminal used to access the internet. Alternately, all these operations may be performed at a single computer such as a desktop system or portable laptop type system.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. A method, comprising:
determining steady state values for daily caloric parameters that conform a person to weight management goal, the daily caloric parameters comprising a daily base metabolic rate (BMR) and a target offset from a daily total daily energy expenditure (TDEE), wherein the TDEE comprises the BMR;
determining an intake shortfall for a plurality of days, the shortfall comprising a sum of daily shortfalls between actual daily offsets and the steady state target offset;
making a recommendation comprising food to be consumed to reduce the shortfall, the recommendation effective to account for a transient change in a hunger response resulting from the consumption of the recommended food;
causing consumption on an up adjustment day; and
determining transient values for at least one of the daily caloric parameters to be used subsequent to the consumption on the up-adjustment day effective to bring an average of the actual daily offsets closer to the steady state target offset.

2. The method of claim 1, wherein the recommendation reduces the shortfall to zero.

3. The method of claim 1, wherein the transient values for at least one of the daily caloric parameters brings the average of the actual daily offsets to the steady state target offset.

4. The method of claim 1, wherein the recommendation comprises a total caloric impact comprising: a caloric value of the recommended food; and a hunger response allotment for a caloric impact of the recommended food on the hunger response.

5. The method of claim 4, wherein the total caloric impact equals the shortfall.

6. The method of claim 4, wherein the hunger response allotment is calculated based on a composition of the recommended food and considers how much the composition will alter a steady state hunger response.

7. The method of claim 6, wherein the calculation is based on an amount of an appetite-stimulating constituent in the recommended food.

8. The method of claim 4, further comprising determining a hunger response allotment based on a composition of food recently consumed by the person.

9. The method of claim 1, further comprising:
determining a target intake for the up-adjustment day, based on a TDEE for the up-adjustment day and the steady state target offset;
determining surplus calories actually consumed above the target intake for the up-adjustment day;
determining a BMR allotment for a caloric impact of the surplus calories on the BMR;
spreading the BMR allotment out over at least one day by a respective daily amount; and
including the respective daily amount in the target intake for a respective day.

10. The method of claim 1, further comprising:
determining a target intake for the up-adjustment day, based on a TDEE for the up-adjustment day and the steady state target offset;
determining surplus calories actually consumed above the target intake for the up-adjustment day;
determining any unmet shortfall by subtracting the surplus calories from the shortfall;
spreading out the unmet shortfall over at least one day and by a respective daily amount, and
including the respective daily amount in target intake for a respective day.

11. The method of claim 1, further comprising:
determining a maximum allowable amount of a restricted constituent; and
selecting food to be consumed in a manner that ensures the maximum allowable amount of the restricted constituent is not exceeded.

12. The method of claim 1, further comprising selecting food to be consumed that is contra-indicated by a diet plan associated with the weight management goal.

13. The method of claim 1, further comprising selecting a diet plan dietary composition, comprising one of: weight loss, weight maintenance, weight gain, and ketogenic.

14. The method of claim 1, further comprising determining a weight of an individual to determine the weight management goal using a body weight device.

15. A method, comprising:
determining a steady state value for a target offset from a daily total daily energy expenditure (TDEE) that conforms a person to a weight management goal;
causing a change in caloric intake for a plurality of days and responsive to the steady state value for the target offset;
determining an intake shortfall for the plurality of days, the shortfall comprising a sum of daily shortfalls between actual daily offsets and the steady state target offset; and
making a recommendation comprising food to be consumed to reduce the shortfall, wherein the recommendation comprises a total caloric impact comprising a caloric value of the food to be consumed and a hunger response allotment for a caloric impact of the recommended food on a hunger response, wherein the hunger response allotment is calculated based on a composition of appetite-stimulating constituents in the recommended food and considers an effect the appetite-stimulating constituents will have on a steady state hunger response.

16. The method of claim 15, further comprising a determining transient value for a target intake to be used subsequent to consumption on an up-adjustment day effective to bring an average of the actual daily offsets closer to the steady state target offset.

17. The method of claim 15, further comprising:
determining surplus calories actually consumed above a steady state target offset from TDEE on an up-adjustment day;
determining any unmet shortfall by subtracting the surplus calories from the shortfall;
spreading out the unmet shortfall over at least one day and by a respective daily amount, and
including the respective daily amount in a target intake for a respective day.

18. The method of claim 15, further comprising:
determining surplus calories actually consumed above a steady state target offset from TDEE on an up-adjustment day;
determining a BMR allotment for a caloric impact of the surplus calories on the BMR;
spreading the BMR allotment out over at least one day by a respective daily amount; and
including the respective daily amount in a target intake for a respective day.

19. A method, comprising:
determining a steady state value for a target offset from a daily total daily energy expenditure (TDEE);
determining an intake shortfall for a plurality of days, the shortfall comprising a sum of daily shortfalls between actual daily offsets and the steady state target offset;
determining surplus calories actually consumed above the steady state target offset from the TDEE for an up-adjustment day;
determining an unmet intake shortfall by subtracting the surplus calories from the shortfall;
spreading out the unmet shortfall over at least one day and by a respective daily amount;
including the respective daily amount in a target intake for a respective day; and
causing consumption of the unmet shortfall.

20. The method of claim 19, further comprising:
determining a BMR allotment for a caloric impact of the food actually consumed on the BMR;
spreading the BMR allotment out over at least one day by a respective daily amount; and
including the respective daily amount in the target intake for a respective day.
